(19) United States
(12) Patent Application Publication
(10) Pub. No.: US 2006/0025711 A1
(43) Pub. Date: Feb. 2, 2006

(54) Functional hand splint

(76) Inventors: Louis Bell, Chester-Le-Street (GB); Tim Platt, Guisborough (GB)

Correspondence Address: MACMILLAN SOBANSKI & TODD, LLC;ONE MARITIME PLAZA FOURTH FLOOR, 720 WATER STREET, TOLEDO, OH, 43604-1619, US

(21) Appl. No.: 11/179,057

(22) Filed: Jul. 11, 2005

(30) Foreign Application Priority Data
Jul. 12, 2004 (GB) 0415538.8

(51) Int. Cl. A61F 5/01; A61F 2/54

(52) U.S. Cl. 602/21; 623/65

(57) ABSTRACT

A hand splint includes a conically shaped grip portion having a first end and a second end. The first and second ends each include a tool/implement holder in each of which a tool/implement may be removably inserted and held.

### BACKGROUND OF THE INVENTION

This invention relates to hand splints and, in particular, to hand splints that allow disabled persons to use tools and implements.

Decreased functionality in the hand, sometimes referred to as “Tight Hand”, affects people of all ages and medical backgrounds, for example, those suffering from rheumatoid arthritis, stroke, cerebral palsy, spasticity, etc.

Current treatment for this condition is a program of physiotherapy focusing on lengthening the tissues in the hand maintained by the use of a cone-shaped splint.

A cone-shaped splint not only maintains the length of the tissues, but also ensures that the hand is in a functional position. One type of cone shaped splint is described in U.S. Pat. No. 5,152,740, where the splint is in the form of an inflatable bladder. Another inflatable hand splint is described in U.S. Pat. No. 5,020,515.

Another type of hand splint is known from U.S. Pat. No. 4,960,114, which describes a hand splint comprising a shaped and oriented grip member and a brace for attachment to the forearm.

Devices in which utensils can be inserted are also known. One such device consists of a ball of high density foam in which an implement can be inserted or attached by means of an adaptor. The device is marketed under the name Dexball™. It is also known to provide foam handles for cutlery, such handles being marketed under the name Selectalite Cutlery™.

One problem with the known splints is that while they serve to maintain the length of the tissues in the hand, when a patient is holding the splint, the whole arm of the hand holding the splint tends to be immobile, unless the patient is working through an exercise with or on the instruction of a physiotherapist.

Another problem with the known splints is that unless instructed, some patients hold the splint with the cone the wrong way around in the hand.

None of the known hand splints provide for a hand to be maintained in a functional position, whilst at the same time using the arm and hand to do work. By facilitating patients to undertake everyday tasks, the ability to use the hand and arm can be increased and, therefore, the patient's independence and quality of life is improved.

It would therefore be desirable to improve hand splints.

### SUMMARY OF THE INVENTION

This invention relates to hand splints and, in particular, to hand splints that allow disabled persons to use tools and implements. The hand splint includes a conically shaped grip portion having a first end and a second end. The first and second ends each include a tool/implement holder in each of which a tool/implement may be removably inserted and held.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1 is a perspective view of a hand splint according to the invention showing a first end of the splint.

FIG. 2 is a perspective view of the hand splint illustrated in FIG. 1 showing a second end thereof.

FIG. 3 is a perspective view of the hand splint illustrated in FIG. 1 with a tool/implement attached thereto.

FIG. 4 is a perspective view of the hand splint illustrated in FIG. 2 with a tool/implement attached thereto.

### DETAILED DESCRIPTION OF THE PREFERRED EMBODIMENT

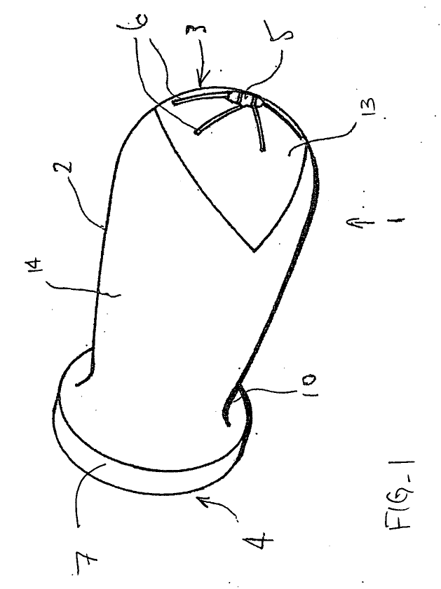

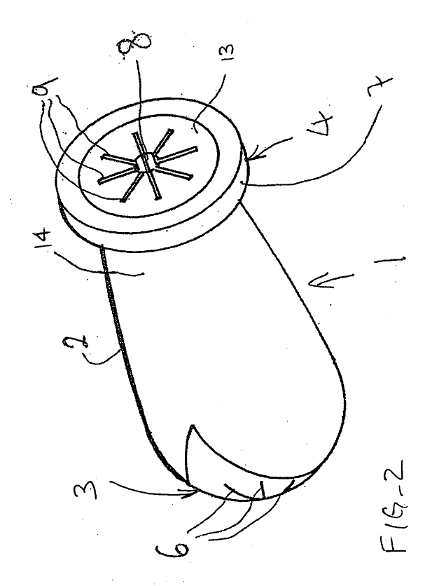

Referring now to FIGS. 1 and 2, there is shown a hand splint 1 including a grip portion 2, a first end 3, and a second end 4. In cross-section, along an axis extending between the first end 3 and the second end 4, the grip portion 2 is generally conical in shape, with edges thereof being provided with curved surfaces.

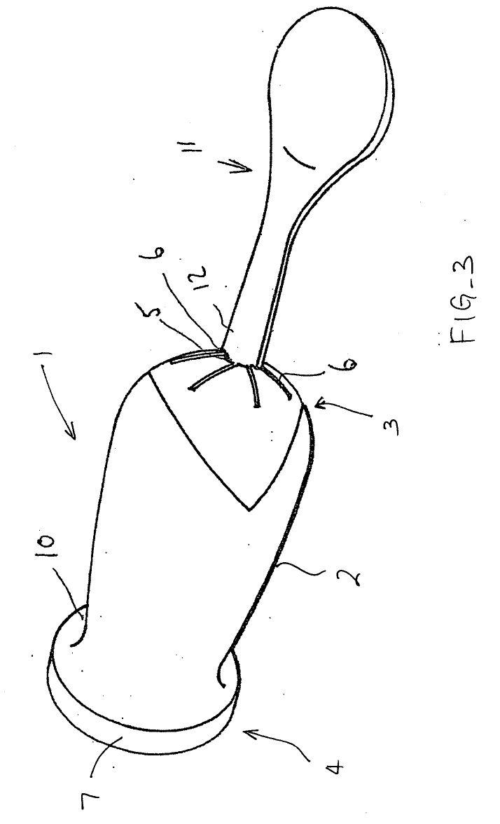

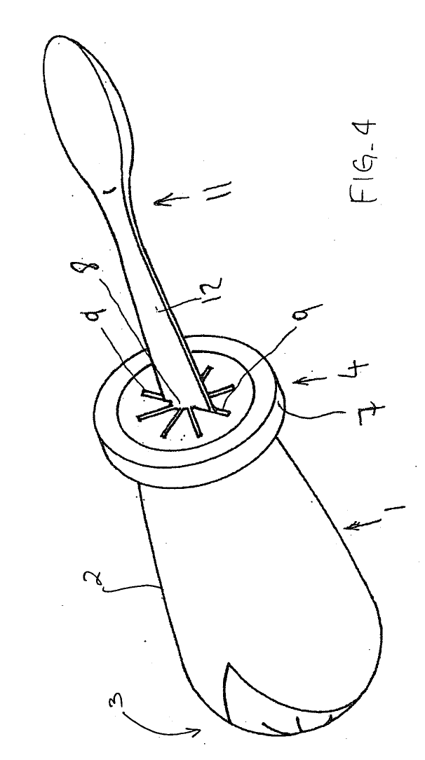

The first end 3 of the hand splint 1 includes a bore 5 having a plurality of slots 6 extending therefrom. The use of these slots 6 will be described in greater detail with reference to FIGS. 3 and 4.

The second end 4 is in the form of a collar 7 of larger diameter than a neck 10 of the grip portion 2 at the point where the two meet.

In use, the hand splint 1 is grasped with the thumb and index finger sitting in the neck 10. The collar 7 is used as a point of reference to assist in the correct location of the splint 1 in the hand and to prevent the splint from slipping through the hand.

As illustrated in FIG. 2, the second end 4 includes a bore 8 having a plurality of slots 9 extending therefrom. The collar 7 forms the outer edge of the second end 4.

FIGS. 3 and 4 illustrate the splint 1 with a tool/implement in the form of a spoon 11 attached thereto. As shown in FIG. 3, the spoon 11 includes a handle 12 that can be inserted into the bore 5 and slots 6 provided in the first end 3 of the splint 1. With the splint 1 gripped in the hand, the radial angle of the spoon 11 with respect to the longitudinal axis of the splint, and therefore of the hand, is fixed. The radial angle of the spoon 11 with respect to the longitudinal axis of the splint 1 may be changed by moving the spoon 11 from one set of opposing slots 6 to another pair of opposing slots 4. As shown in FIG. 4, the spoon 11 may be located in the second end 4 of the splint 1, the spoon handle 12 being inserted into the bore 8 and opposing slots 9.

As shown in FIGS. 1 and 2, the splint 1 can include a soft inner core 13 surrounded by a hard outer shell 14. The splint 1 is made of polyurethane, with the hard outer shell 14 being formed by the moulding process. Alternatively, the splint 1 could be formed from a soft inner core 13 of a first material, such as polyurethane and a separate hard outer shell 14 of a mouldable plastics material.

In use, the splint 1 is held with the thumb and index finger sitting in the neck 10. By inserting tools or implements in the splint at different angles (by selecting which pair of opposing slots the tool is located in), and by inserting tools or implements into different ends of the splint, exercises can be aimed at developing different arm movements, muscles, and tissues.

In FIGS. 3 and 4, the spoon 11 is inserted into the splint to facilitate self-feeding. Self-feeding as an exercise develops extension at the elbow and the wrist. Furthermore self-feeding is a requirement for independence.

In general, patients with spasticity hold their arms to their chests. In order for such patients to be able to feed themselves, they must be able to move their arms away from their chests. By inserting a tool or implement into the first end of the hand splint 1, this movement can be encouraged because to use the tool or implement the arm must be moved away from the chest. A suitable exercise in this situation would involve inserting a crayon into the first end 3 of the hand splint 1. The patient must then extend the arm away from the chest in order to draw.

A significant advantage of the present invention is that its use does not immobilize the arm. In fact, by using tools or implements in conjunction with the splint, patients are encouraged to use the tools or implements, i.e. undertake functional activity, thereby speeding the development of arm movements, muscles and tissues.

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope.

What is claimed is:

1. A hand splint comprising:
a conically shaped grip portion having a first end and a second end,
wherein the first and second ends each include a tool/implement holder in each of which a tool/implement may be removably inserted and held.

2. A hand splint according to claim 1, wherein the second end is in the form of a collar extending radially and axially of the grip portion.

3. A hand splint according to claim 1, wherein the first end is rounded.

4. A hand splint according to claim 3, wherein the first end is substantially hemi-spherical.

5. A hand splint according to claim 1, wherein the tool/implement holder provides for a tool to be inserted and held in the splint at a plurality of orientations relative to the grip portion.

6. A hand splint according to claim 5, wherein the tool/implement holder provides for the tool/implement to be inserted and held in the splint at a plurality of spaced apart orientations, each orientation lying on a different radial axis of the splint.

7. A hand splint according to claim 5, wherein the tool/implement holder is provided with a plurality of slots.

8. A hand splint according to claim 7, wherein the slots bisect each other and the point of bisection lies substantially on a central longitudinal axis of the splint.

9. A hand splint according to claim 1, wherein the tool holder includes a bore.

10. A hand splint according to claim 9, wherein the tool/implement holder provides a plurality of slots, and wherein the bore extends into the splint along a substantially central longitudinal axis of said splint.

11. A splint according to claim 9, wherein the tool/implement holder provides a plurality of slots, and wherein the slots are arranged in pairs, each slot of a pair extending to an opposite side of the bore and the slots of a pair being aligned with each other.

12. A hand splint according to claim 1, wherein the splint comprises a soft inner core and a hard outer shell.

13. A hand splint according to claim 12, wherein the soft inner core and hard outer shell are both formed from polyurethane.

14. A hand splint according to claim 13, wherein the soft inner core and the hard outer shell are formed in the same moulding process.

15. A hand splint according to claim 12, wherein the soft inner core and hard outer shell are formed from separately, the hard outer shell being attached to the soft inner core.
